(19) United States
(12) Patent Application Publication
(10) Pub. No.: US 2020/0181678 A1
(43) Pub. Date: Jun. 11, 2020

(54) Cell-Based Reporter Assay for Live Virus Vaccines

(71) Applicant: Merck Sharp & Dohme Corp., Rahway, NJ (US)

(72) Inventors: BRIAN K. MEYER, NEW BRITAIN, PA (US); AKHILESH BHAMBHANI, DOYLESTOWN, PA (US); JEFFREY T. BLUE, TELFORD, PA (US)

(73) Assignee: Merck Sharp & Dohme Corp., Rahway, NJ (US)

(21) Appl. No.: 16/468,798

(22) PCT Filed: Dec. 11, 2017
(86) PCT No.: PCT/US2017/065501

§ 371 Date: Jun. 12, 2019

Related U.S. Application Data
(60) Provisional application No. 62/434,675, filed on Dec. 15, 2016.

(51) Int. Cl. C12Q 1/66; C12N 15/85; C12Q 1/02; G01N 21/64

(52) U.S. Cl. CPC C12Q 1/66; G01N 21/6428; C12Q 1/02; C12N 15/85

(57) ABSTRACT

The present invention relates to a cell-based reporter assay for determining viral potency comprising A) transfecting cells maintained in media with a promoter-reporter construct and generating reporter enzyme within the cells; B) infecting cells with a live virus or live virus vaccine wherein reporter enzyme is released into media; and C) measuring reporter enzyme intensity in media.

### BACKGROUND OF THE INVENTION

The industry standard for determining viral potency of live virus vaccines is the plaque potency assay (Dulbecco and Vogt, 1953, Cooper, 1961, Hartley and Rowe, 1963, Baer and Kehn-Hall, 2014). This assay is based on plaque identification and is low throughput, requires significant resources, and has high variability. Thus, an assay for determining viral potency of live virus vaccines that is fast, easy to use, and has low variability would be an asset for the development of live virus vaccine candidates.

One such assay is a cell-based reporter assay. Cell-based reporter assays exist that may be used to measure the viral potency of live viruses or live virus vaccines (Niles et. al., 2013, Li et. al., 2009). These cell-based reporter assays may employ commercial kits, which include, CellTiter-Glo® and Viral ToxGlo™ (Promega, Madison, Wis.). Both kits rely on measuring ATP inside living cells. The Nano-Glo® assay, which is another cell-based reporter assay, utilizes lysis buffer and furimazine (substrate), for detection of NanoLuc® enzyme introduced into living cells (Khuc et. al., 2016, Masser et. al., 2016). These cell-based reporter assays, which measure molecules and/or enzymes within cells, may have drawbacks which include plate bias as well as edge effects (different potency values on the edges of the plate as compared to the centered samples) which reduces the number of wells that can be used in the plate. For example, if edge effects are observed, these wells will not be used, thereby reducing the total number of wells that are available for the assay.

### SUMMARY OF THE INVENTION

The invention is directed to a high-throughput, cell-based reporter assay in a multi-well (96-well or 386-well) format for determining the viral potency of live virus vaccines, with an emphasis on rVSV-ΔG vaccines. The assay utilizes Vero E6 cells that contain a stably integrated CMV promoter-NanoLuc® construct and are capable of expressing the NanoLuc® enzyme within the cells. When these Vero E6 cells are infected by a vaccine candidate such as rVSV-ΔG-ZEBOV-GP (recombinant vesicular stomatitis virus-Zaire Ebolavirus), NanoLuc® enzyme is released into the media where its intensity is measured. This is the first known cell-based reporter assay for evaluating the potency of rVSV-ΔG vaccines. Furthermore, the multi-well format allows for the possibility of integration with a high-throughput dispensing robot for increased assay turnover and capacity. Automation can allow an increase in the number of replicates per sample, thereby substantially reducing the assay variability.

### BRIEF DESCRIPTION OF THE DRAWINGS

FIG. 1. NanoLuc® enzyme levels in the media of Vero E6/CMV-NanoLuc® cell cultures concomitantly increase with increasing titers of rVSV-ΔG-ZEBOV-GP.

FIG. 2. Evaluation of NanoLuc® enzyme expression at 24, 40, and 48 hours of infection with rVSV-ΔG-ZEBOV-GP using Vero E6/CMV-NanoLuc® cells at a density of 20,000, 40,000 and 60,000 cells/well.

FIG. 3. Optimization of rVSV-ΔG-ZEBOV-GP infection time and cell count/well with Vero E6/CMV-NanoLuc® cells.

FIG. 4. The Vero E6/CMV-NanoLuc® assay and the plaque potency assay demonstrate concordance using rVSV-ΔG-ZEBOV-GP.

FIG. 5. The Vero E6/CMV-NanoLuc® cell line is stability-indicating for rVSV-ΔG-ZEBOV-GP.

FIG. 6. Comparison of the NanoLuc® and CellTiterGlo® assays.

FIG. 7. An 11-point curve generated with Vero E6/CMV-NanoLuc® infected with rVSV-ΔG-ZEBOV-GP.

FIG. 8. Plot of percent response data for positive control.

### DETAILED DESCRIPTION OF THE INVENTION

The present invention relates to a cell-based reporter assay for determining viral potency comprising A) transfecting cells maintained in media with a promoter-reporter construct and generating reporter enzyme within the cells; B) infecting cells with a live virus or live virus vaccine wherein reporter enzyme is released into media; and C) measuring reporter enzyme intensity in media.

In an embodiment, the present invention relates to an assay for determining viral potency wherein the cells are selected from insect, animal, or human cells.

In an embodiment, the present invention relates to an assay for determining viral potency wherein the cells are selected from Vero, Vero E6, MeWo, HEK293, CHO, MC3T3, DU145, H295R, HeLa, KBM-7, LNCaP, MCF-7, MDA-MB-468, PC3, SaOS-2, SH-SY5Y, T47D, THP-1, U87, NCI60, GH, PC12, BY-2, MDCK, A6, AB9, ARPE19, and MRC-5 cells and any modifications thereof.

In an embodiment, the present invention relates to an assay for determining viral potency wherein the cells are selected from Vero E6 cells.

In an embodiment, the present invention relates to an assay for determining viral potency wherein the promoter is any constitutive promoter.

In an embodiment, the present invention relates to an assay for determining viral potency wherein the promoter is the CMV promoter.

In an embodiment, the present invention relates to an assay for determining viral potency wherein the reporter is the NanoLuc® enzyme or the Luc2 enzyme.

In an embodiment, the present invention relates to an assay for determining viral potency wherein the reporter is the NanoLuc® enzyme.

In an embodiment, the present invention relates to an assay for determining viral potency wherein the reporter is SEQ ID 4 or SEQ ID 5.

In an embodiment, the present invention relates to an assay for determining viral potency wherein the reporter is SEQ ID 4.

In an embodiment, the present invention relates to an assay for determining viral potency wherein the live virus or live virus vaccine is selected from viruses with any single-stranded RNA genome that is negative-sense or positive-sense, and viruses with double-stranded RNA or double-stranded DNA genomes.

In an embodiment, the present invention relates to an assay for determining viral potency wherein the live virus is selected from a filovirus, herpesvirus, paramyxovirus, arenavirus, adenovirus, rhabdovirus, flavivirus, and orthomyxovirus.

In an embodiment, the present invention relates to an assay for determining viral potency wherein the live virus vaccine is selected from a filovirus, herpesvirus, paramyxovirus, arenavirus, adenovirus, rhabdovirus, flavivirus, and orthomyxovirus vaccine.

In an embodiment, the present invention relates to an assay for determining viral potency wherein the live virus is a filovirus.

In an embodiment, the present invention relates to an assay for determining viral potency wherein the live virus vaccine is a filovirus vaccine.

In an embodiment, the present invention relates to an assay for determining viral potency wherein the live virus is a rVSV-ΔG virus.

In an embodiment, the present invention relates to an assay for determining viral potency wherein the live virus vaccine is selected from a rVSV-ΔG virus vaccine.

In an embodiment, the present invention relates to an assay for determining viral potency wherein the live virus vaccine is selected from a recombinant vaccine, a therapeutic vaccine, or selected from a vaccine that is from an attenuated virus, an enveloped virus, or recombinant virus.

In an embodiment, the present invention relates to an assay for determining the potency of a lipid nanoparticle vaccine, or a lipid nanoparticle vaccine containing RNA, for example, small interfering RNA (siRNA) or messenger RNA (mRNA).

In an embodiment, the present invention relates to an assay for determining viral potency wherein the live virus is a rVSV-ΔG virus containing a glycoprotein derived from another virus.

In an embodiment, the present invention relates to an assay for determining viral potency wherein the live virus vaccine is a rVSV-ΔG virus vaccine containing a glycoprotein derived from another virus.

In an embodiment, the present invention relates to an assay for determining viral potency wherein the media is in liquid form, gel form or solid form.

In an embodiment, the present invention relates to an assay for determining viral potency wherein the media is in liquid form or gel form.

In an embodiment, the present invention relates to an assay for determining viral potency wherein the media is in liquid form.

In an embodiment, the present invention relates to an assay for determining viral potency wherein the assay is run in a multi-well format.

In an embodiment, the present invention relates to an assay for determining viral potency wherein the promoter-reporter construct is stably integrated.

In an embodiment, the present invention relates to an assay for determining viral potency wherein the assay is run in a multi-well format which comprises the use of 96- or 384-well plates.

In another embodiment, the present invention is a stable Vero E6 cell line containing a CMV promoter, or another constitutive promoter fused to the NanoLuc® gene.

In another embodiment, the present invention is a stable Vero E6 cell line containing a reporter construct that is used to measure the enzyme levels following infection by a live virus or live virus vaccine.

In another embodiment, the present invention is a kit comprising a cell line that contains a stably integrated promoter-reporter construct, a plate, and culture media.

In another embodiment, the present invention is a kit comprising a cell line that contains a promoter-reporter construct for determining viral potency in media, a 96-well plate, and culture media containing fetal bovine serum for growing the cells.

In another embodiment, the present invention relates to a cell-based reporter assay for determining viral potency comprising A) transfecting Vero E6 cells maintained in media with a promoter-reporter construct wherein the promoter is CMV and the reporter is NanoLuc® and generating the NanoLuc® enzyme within the Vero E6 cells; B) infecting the Vero E6 cells with a live virus which is rVSV-ΔG backbone; C) incubating the cells for 1-3 days at approximately 37° C. and 5% CO₂ wherein the NanoLuc® enzyme is released into the media; D) removing media; E) mixing media with substrate, and F) measuring emitted light.

In another embodiment, the present invention relates to a cell-based reporter assay for determining viral potency comprising A) transfecting Vero E6 cells maintained in media with a promoter-reporter construct wherein the promoter is CMV and the reporter is NanoLuc® enzyme and generating the NanoLuc® enzyme within the Vero E6 cells; B) infecting the Vero E6 cells with a live virus which has a rVSV-ΔG backbone; C) incubating the cells for 1-3 days at approximately 37° C. and 5% CO₂ wherein the NanoLuc® enzyme is released into the media if the cells are lysed by the virus; D) removing media; E) mixing media with substrate, and F) measuring emitted light.

In another embodiment, the present invention relates to a cell-based reporter assay for determining viral potency comprising A) transfecting Vero E6 cells maintained in media with a promoter-reporter construct wherein the promoter is CMV and the reporter is NanoLuc® and generating the NanoLuc® enzyme within the Vero E6 cells; B) infecting the Vero E6 cells with a live virus vaccine which is the rVSV-ΔG backbone; C) incubating the cells for 1-3 days at approximately 37° C. and 5% CO₂ wherein the NanoLuc® enzyme is released into the media; D) removing media; E) mixing media with substrate, and F) measuring emitted light.

In another embodiment, the present invention relates to a cell-based reporter assay for determining viral potency comprising A) transfecting Vero E6 cells maintained in media with a promoter-reporter construct wherein the promoter is CMV and the reporter is NanoLuc® enzyme and generating the NanoLuc® enzyme within the Vero E6 cells; B) infecting the Vero E6 cells with a live virus vaccine which comprises rVSV-ΔG; C) incubating the cells for 1-3 days at approximately 37° C. and 5% CO₂ wherein the NanoLuc® enzyme is released into the media if the cells are lysed by the virus vaccine; D) removing media; E) mixing media with substrate, and F) measuring emitted light.

In another embodiment, the present invention relates to an assay to determine viral potency wherein the assay is performed in a 96-well plate or a 384-well plate format with at least triplicate sample runs per assay.

In another embodiment, the present invention relates to an assay to determine viral potency wherein the assay variability is less than or equal to about 50%.

In another embodiment, the present invention relates to an assay to determine viral potency wherein the assay variability is less than or equal to about 29%.

In another embodiment, the present invention relates to an assay to determine viral potency wherein the assay rep-to-rep variability is less than equal to about 22%.

In another embodiment, the present invention relates to an assay to determine viral potency wherein the assay plate-to-plate variability is less than equal to about 18%.

In another embodiment, the present invention relates to an assay to determine viral potency wherein the assay run-to-run variability is less than equal to about 5%.

In another embodiment, the present invention relates to a cell-based reporter assay for determining viral potency comprising A) transfecting cells maintained in media with a promoter-reporter construct and generating reporter enzyme within the cells; B) infecting cells with a live virus or live virus vaccine wherein reporter enzyme is released into media if the cells are lysed by the virus; and C) measuring reporter enzyme intensity in media.

### Definitions

The term “3σ” refers to the range in which the value of the positive control can fall to determine if the assay run is valid. The 3σ value is determined by taking the mean +/−3 standard deviations.

A “coding sequence” is known to those skilled in the art, and means a nucleotide sequence that, when transcribed and translated, in the case of this invention, results in the production of a protein product. A coding sequence, such as the NanoLuc® reporter gene (the “reporter” construct) as described in this invention, is “linked,” “associated with” or “under the control of” a transcriptional and translational control sequence, such as a promoter, such as the CMV promoter. These sequences are present in an isolated host cell, and these sequences direct transcription of the coding sequence by RNA polymerase.

“derivatives”

The term “Ebola virus” is known to those skilled in the art, and means a single-stranded RNA virus that causes ebola virus disease (EVD), which is characterized by electrolyte losses, and suppression of the immune system, among other characteristics. EVD can result in fatality or be asymptomatic.

The term “Ebola virus vaccine” is known to those skilled in the art, and means a vaccine that prevents infection by the Ebola virus.

The terms “express” and “expression” mean allowing or causing the information in a gene, RNA or DNA sequence to become manifest; for example, producing a protein by activating the cellular functions involved in transcription and translation of a corresponding gene. A DNA sequence is expressed in or by a cell to form an “expression product” such as an RNA (e.g., mRNA) or a protein. The expression product itself may also be said to be “expressed” by the cell.

The term “filovirus” is known to those skilled in the art, and means the family of viruses referred to as Filoviridae. Filoviruses have a single-stranded, negative-sense RNA genome, and their virions are characterized by being filamentous. Examples of filoviruses include Ebola and Marburg viruses. These viruses cause viral hemorrhagic fevers.

The term “live virus” is known to those skilled in the art, and means any virus that has the ability to replicate, either in cell culture, in embryonated eggs, or when administered into humans.

The term “live virus vaccine” is known to those skilled in the art, and means any vaccine that contains an attenuated live virus. Attenuated viruses cannot themselves cause disease. Viruses may be attenuated by growth in cell culture at 30° C., for example. When the “attenuated” virus is injected into a human, for example as part of a “live virus vaccine”, with a 37° C. body temperature, some replication may occur, however the virus cannot replicate sufficiently to cause disease.

The term “media” is known to those skilled in the art, and means cell culture material used to support growth. The media can be a liquid form, a gel form or a solid form.

The term “modifications” refers to any change or alteration of an immortalized cell line. The change or alteration may refer to the insertion of a sequence of DNA into the genome which is carried to progeny during replication.

The term “NanoLuc®” refers to a luminescent enzyme that was derived from a deep-sea shrimp and was codon-optimized for use in mammalian systems using a novel substrate, furimazine. The glow-type luminescence of the NanoLuc® enzyme has a specific activity that is 150-fold greater than firefly or renilla luciferases. NanoLuc® is a product from Promega (Madison, Wis.).

The term “nucleic acid” is known to those skilled in the art, and means biopolymers, or large biomolecules, which include DNA (deoxyribonucleic acid) and RNA (ribonucleic acid), and are made from monomers known as nucleotides. Each nucleotide has three components: a 5-carbon sugar, a phosphate group, and a nitrogenous base. If the sugar is deoxyribose, the polymer is DNA. If the sugar is ribose, the polymer is RNA. When all three components are combined, they form a nucleotide.

The term “parallelism” refers to the ratio of the slope of the curve of the reference standard to the sample. Parallelism is a means to compare samples.

The term “positive control” herein refers to a frozen stock of rVSV-ΔG-ZEBOV-GP in a buffer that is different than the reference standard with a titer of 2E7 PFU/mL, which is half of the reference standard. The positive control is utilized to verify the assay run is valid.

A “promoter” is known to those skilled in the art, and means a region of DNA that initiates transcription of a particular gene. For example, the cytomegalovirus (CMV) promoter, exemplified herein, initiates transcription of the NanoLuc® gene. A “constitutive promoter” is known to those skilled in the art, and means a promoter that allows for continual transcription of its associated gene.

The term “rVSV” means a recombinant vesicular stomatitis virus. rVSV strains are used for vaccines in humans. rVSV comprises the (genetic) removal of the native surface glycoprotein of VSV, which is termed rVSV-ΔG, or absence of the native glycoprotein and replacement with the surface glycoprotein from another virus, such as Zaire Ebola.

The term “rVSV-glycoprotein-containing vaccine” means a recombinant vesicular stomatitis virus (rVSV) in which the native VSV surface glycoprotein sequence is removed from the VSV genome and replaced with a glycoprotein sequence from another virus, such as the Zaire Ebola strain. The recombinant VSV will then express the surface glycoprotein that was inserted into its genome on its surface. The rVSV may then be used as a vaccine because when introduced into the body, an immune response is elicited against the heterologous surface glycoprotein. By an immune response being elicited against the surface glycoprotein from another virus, protection against that virus is achieved. Examples of rVSV viruses include rVSV-ΔG-ZEBOV-GP.

The term “rVSVs-ΔG virus” means any recombinant vesicular stomatitis virus with the surface glycoprotein removed (delta symbol-G for glycoprotein) and replaced with a surface glycoprotein from another virus (for example Zaire Ebolavirus).

The term “rVSVs-ΔG vaccines” means any vaccine made with recombinant vesicular stomatitis virus with the surface glycoprotein removed (delta symbol-G for glycoprotein) and replaced with a surface glycoprotein from another virus (for example Zaire Ebolavirus). The term rVSV-ΔG and the term “rVSV-ΔG backbone” are used interchangably.

The term “backbone” refers to the ability of the rVSV-ΔG virus to be used as a platform for incorporation of surface glycoproteins from other viruses.

The term “rVSV-ΔG-ZEBOV-GP” means a recombinant vesicular stomatitis virus with the native surface glycoprotein removed (indicated by the delta symbol) and replaced with the ZEBOV-GP, or Zaire Ebolavirus surface glycoprotein.

The term “reference standard” refers to frozen stock solution consisting of rVSV-ΔG-ZEBOV-GP in a buffer that has a titer of 4E7 plaque forming units (PFU)/mL. The reference standard is created by diluting the 4E7 PFU/mL stock with serial dilutions at a ratio of 1:4.

The term “reporter construct” is known to those skilled in the art, and means any DNA vector or plasmid that contains a promoter that drives the transcription of a gene that results in the eventual translation into a protein that is used as a “reporter.” The term “reporter” indicates that a cellular event, infection, etc. is “reported.” The “reporter” is typically an enzyme that can be measured (detected) by means of instrumentation, one example being a luminometer. Examples of “reporters” include the enzymes NanoLuc® (derived from a deep sea shrimp), Luc and Luc2 (derived from the firefly), and others well known in the art.

The term “stability sample” refers to a stock of rVSV-ΔG-ZEBOV-GP in a buffer that was incubated for three days at 25 C and has a titer of 4E7 PFU/mL. The stability sample was used in the Variance Component Analysis (VCA).

The term “stability indicating” refers to the ability of an assay to detect changes in the stability of a virus or other substance.

The term “stably integrated” (as denoted herein with the term “promoter-reporter construct”) is known to those skilled in the art and means a fragment or sequence of DNA that is incorporated into a host genome and replicates as the cell replicates (i.e. is carried to progeny).

The term “transfection” is known to those skilled in the art, and means the introduction of a nucleic acid into a cell. These terms may refer to the introduction of a nucleic acid encoding CMV-NanoLuc® into a cell. The introduced gene or sequence may be called a “clone”. A host cell that receives the introduced DNA or RNA has been “transformed” and is a “transformant” or a “clone”. The DNA or RNA introduced to a host cell can come from any source, including cells of the same genus or species as the host cell, or cells or virus of a different genus or species.

The terms “vector”, “cloning vector” and “expression vector” are known to those skilled in the art, and means the vehicle (e.g., a plasmid) by which a DNA or RNA sequence can be introduced into a host cell, so as to transform the host and, optionally, promote expression and/or replication of the introduced sequence.

The term “viral potency” is known to those skilled in the art, and means the ability of a virus to infect a cell. A high viral potency means there are sufficient quantities of virus that are capable of infecting cells. A low viral potency means there are low or insufficient quantities of infectious virus to infect cells. A high viral potency is observed with viruses that are in quantities sufficient to infect many cells, and themselves are intact, infectious particles. It is possible to have high quantities of virus, however the potency may be low if the majority of viruses are not infectious. A low viral potency is typically observed due to low amounts of intact, infectious virus being present.

### Molecular Biology

In the present invention, molecular biology techniques were utilized, including recombinant DNA and microbiological techniques, within the skill of the art. These techniques are described in the following literature references. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook, et al., 1989”); DNA Cloning: A Practical Approach, Volumes I and II (D.N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

Host cells that can be used in a screening assay of the present invention include Vero E6 (American Type Culture Collection (ATCC), under number C1008, or CRL-1586).

### CMV-NanoLuc®

The present invention provides a fusion of the CMV promoter with the NanoLuc® reporter gene. This sequence was placed into the host cells (e.g., host cells that are discussed herein) comprising the fusion of the CMV promoter with the NanoLuc gene and methods of use thereof, e.g., as is discussed herein.

In one embodiment, the following nucleotide sequence (SEQ ID NO:1) comprising a fusion of the CMV promoter with the NanoLuc reporter gene was inserted into the pGL4.17 vector (Promega, Madison, Wis.). The restriction nuclease site at the 5′ end is HindIII and the restriction nuclease site at the 3′ end is Mfel:

(SEQ ID NO: 1)
5′-AAGCTTATTGATTATTGACTAGTTATTAATAGTAATCAATTACGGGG
TCATTAGTTCATACCCATATATGGAGTTCCGCGTTACATAACTTACGGTA
AATGGCCCGCCTGGCTGACCGCCCAACGACCCCCGCCCATTGACGTCAAT
AATGACGTATGTTCCCATAGTAACGCCAATAGGGACTTTCCATTGACGTC
AATGGGTGGAGTATTTACGGTAAACTGCCCACTTGGCAGTACATCAAGTG
TATCATATGCCAAGTACGCCCCCTATTGACGTCAATGACGGTAAATGGCC
CGCCTGGCATTATGCCCAGTACATGACCTTATGGGACTTTCCTACTTGGC
AGTACATCTACGTATTAGTCATCGCTATTACCATGGTGATGCGGTTTTGG
CAGTACATCAATGGGCGTGGATAGCGGTTTGACTCACGGGGATTTCCAAG
TCTCCACCCCATTGACGTCAATGGGAGTTTGTTTTGGCACCAAAATCAAC
GGGACTTTCCAAAATGTCGTAACAACTCCGCCCCATTGACGCAAATGGGC
GGTAGGCGTGTACGGTGGGAGGTCTATATAAGCAGAGCTCTCTGGCTAAC
TAGAGAACCCACTGCTTACTGGCTTATCGAAATTAATACGACTCACTATA
GGGGCAATCCGGTACTGTTGGTAAAGCCACCATGGTCTTCACACTCGAAG
ATTTCGTTGGGGACTGGCGACAGACAGCCGGCTACAACCTGGACCAAGTC
CTTGAACAGGGAGGTGTGTCCAGTTTGTTTCAGAATCTCGGGGTGTCCGT
AACTCCGATCCAAAGGATTGTCCTGAGCGGTGAAAATGGGCTGAAGATCG
ACATCCATGTCATCATCCCGTATGAAGGTCTGAGCGGCGACCAAATGGGC
CAGATCGAAAAAATTTTTAAGGTGGTGTACCCTGTGGATGATCATCACTT
TAAGGTGATCCTGCACTATGGCACACTGGTAATCGACGGGGTTACGCCGA
ACATGATCGACTATTTCGGACGGCCGTATGAAGGCATCGCCGTGTTCGAC
GGCAAAAAGATCACTGTAACAGGGACCCTGTGGAACGGCAACAAAATTAT
CGACGAGCGCCTGATCAACCCCGACGGCTCCCTGCTGTTCCGAGTAACCA
TCAACGGAGTGACCGGCTGGCGGCTGTGCGAACGCATTCTGGCGTAAGGC
CGCGACTCTAGAGTCGGGGCGGCCGGCCGCTTCGAGCAGACATGATAAGA
TACATTGATGAGTTTGGACAAACCACAACTAGAATGCAGTGAAAAAAATG
CTTTATTTGTGAAATTTGTGATGCTATTGCTTTATTTGTAACCATTATAA
GCTGCAATAAACAAGTTAACAACAACAATTG-3′

The CMV immediate/early enhancer/promoter is known in the art. The nucleotide sequence is as follows:

(SEQ ID NO: 2)
5′-ATTATTGACTAGTTATTAATAGTAATCAATTACGGGGTCATTAGTTC
ATAGCCCATATATGGAGTTCCGCGTTACATAACTTACGGTAAATGGCCCG
CCTGGCTGACCGCCCAACGACCCCCGCCCATTGACGTCAATAATGACGTA
TGTTCCCATAGTAACGCCAATAGGGACTTTCCATTGACGTCAATGGGTGG
AGTATTTACGGTAAACTGCCCACTTGGCAGTACATCAAGTGTATCATATG
CCAAGTACGCCCCCTATTGACGTCAATGACGGTAAATGGCCCGCCTGGCA
TTATGCCCAGTACATGACCTTATGGGACTTTCCTACTTGGCAGTACATCT
ACGTATTAGTCATCGCTATTACCATGGTGATGCGGTTTTGGCAGTACATC
AATGGGCGTGGATAGCGGTTTGACTCACGGGGATTTCCAAGTCTCCACCC
CATTGACGTCAATGGGAGTTTGTTTTGGCACCAAAATCAACGGGACTTTC
CAAAATGTCGTAACAACTCCGCCCCATTGACGCAAATGGGCGGTAGGCGT
GTACGGTGGGAGGTCTATATAAGCAGAGCTC-3′

In an embodiment of the invention, the NanoLucR enzyme comprises the following nucleotide and amino acid sequence:

### Nucleotide

(SEQ ID NO: 3)
5′-ATGGTCTTCACACTCGAAGATTTCGTTGGGGACTGGCGACAGACAGC
CGGCTACAACCTGGACCAAGTCCTTGAACAGGGAGGTGTGTCCAGTTTGT
TTCAGAATCTCGGGGTGTCCGTAACTCCGATCCAAAGGATTGTCCTGAGC
GGTGAAAATGGGCTGAAGATCGACATCCATGTCATCATCCCGTATGAAGG
TCTGAGCGGCGACCAAATGGGCCAGATCGAAAAAATTTTTAAGGTGGTGT
ACCCTGTGGATGATCATCACTTTAAGGTGATCCTGCACTATGGCACACTG
GTAATCGACGGGGTTACGCCGAACATGATCGACTATTTCGGACGGCCGTA
TGAAGGCATCGCCGTGTTCGACGGCAAAAAGATCACTGTAACAGGGACCC
TGTGGAACGGCAACAAAATTATCGACGAGCGCCTGATCAACCCCGACGGC
TCCCTGCTGTTCCGAGTAACCATCAACGGAGTGACCGGCTGGCGGCTGTG
CGAACGCATTCTGGCGTAA-3′

### Amino Acid

MVFTLEDFVGDWRQTAGYNLDQVLEQGGVSSLFQNLGVSVTPIQRIVLSGENGLKIDIH VIIPYEGLSGDQMGQIEKIFKVVYPVDDHHFKVILHYGTLVIDGVTPNMIDYFGRPYEGI AVFDGKKITVTGTLWNGNKIIDERLINPDGSLLFRVTINGVTGWRLCERILA (SEQ ID NO: 4)

In an embodiment of the invention, the Luc2 enzyme comprises the following amino acid sequence:

(SEQ ID NO: 5)
MEDAKNIKKGPAPFYPLEDGTAGEQLHKAMKRYALVPGTIAFTDAHIEVD
ITYAEYFEMSVRLAEAMKRYGLNTNHRIVVCSENSLQFFMPVLGALFIGV
AVAPANDIYNERELLNSMGISQPTVVFVSKKGLQKILNVQKKLPIIQKII
IMDSKTDYQGFQSMYTFVTSHLPPGFNEYDFVPESFDRDKTIALIMNSSG
STGLPKGVALPHRTACVRFSHARDPIFGNQIIPDTAILSVVPFHHGFGMF
TTLGYLICGFRVVLMYRFEEELFLRSLQDYKIQSALLVPTLFSFFAKSTL
IDKYDLSNLHEIASGGAPLSKEVGEAVAKRFHLPGIRQGYGLTETTSAIL
ITPEGDDKPGAVGKVVPFFEAKVVDLDTGKTLGVNQRGELCVRGPMIMSG
YVNNPEATNALIDKDGWLHSGDIAYWDEDEHFFIVDRLKSLIKYKGYQVA
PAELESILLQHPNIFDAGVAGLPDDDAGELPAAVVVLEHGKTMTEKEIVD
YVASQVTTAKKLRGGVVFVDEVPKGLTGKLDARKIREILIKAKKGGKIAV

### Description and Results

A stable cell line, Vero E6/CMV-NanoLuc® (termed JM-1), was developed at Merck & Co., West Point, Pa. Vero E6 cells were derived from African Green Monkey epithelial kidney cells, and are an adherent cell line (ATCC, Manassas, Va.). This cell line was generated by inserting a DNA construct containing the cytomegalovirus (CMV) immediate/early enhancer/promoter in tandem with the NanoLuc® gene (Promega) into the genome of Vero E6 cells. This was accomplished by cloning a fragment containing the CMV immediate/early enhancer/promoter fused to the NanoLuc® gene into a plasmid, and transfecting Vero E6 cells with this construct. Antibiotic selection with geneticin (G418) was utilized to obtain polyclones that contained the stably integrated CMV-NanoLuc® construct. A subsequent cell clone was isolated that had low/background expression of NanoLuc® in the media.

The Vero E6/CMV-NanoLuc® cells constitutively express the NanoLuc® enzyme. This enzyme accumulates in the cytoplasm of the cell and a small fraction is also detected in the culture media. The NanoLuc® enzyme was isolated from a deep sea shrimp and was further optimized to emit light that is approximately 100-fold higher in intensity than firefly luciferase (Promega). The substrate for the NanoLuc® enzyme is furimazine, and the light emitted is measured on a luminometer.

The Vero E6/CMV-NanoLuc® cell line (JM-1), was used to evaluate the viral potency of rVSV-ΔG-ZEBOV-GP in experimental studies. Following infection of the Vero E6/CMV-NanoLuc® cells with rVSV-ΔG-ZEBOV-GP, the cells are believed to lyse and release the NanoLuc® enzyme into the cell culture media. The media is then collected and assayed for the presence of the NanoLuc® enzyme. The assay consists of first infecting the cells with rVSV-ΔG-ZEBOV-GP, incubating for 2 days at 37° C., 5% CO₂, removing a portion of the culture media which contains the NanoLuc® enzyme, mixing it with its substrate, furimazine, followed by measurement of the light emitted by the NanoLuc enzyme with an instrument (e.g. a luminometer). The portion, or volume, of the culture media that is removed is not a critical parameter for the assay.

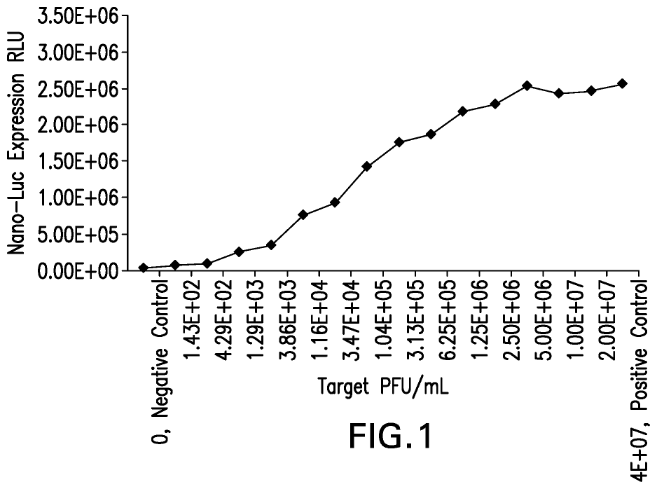

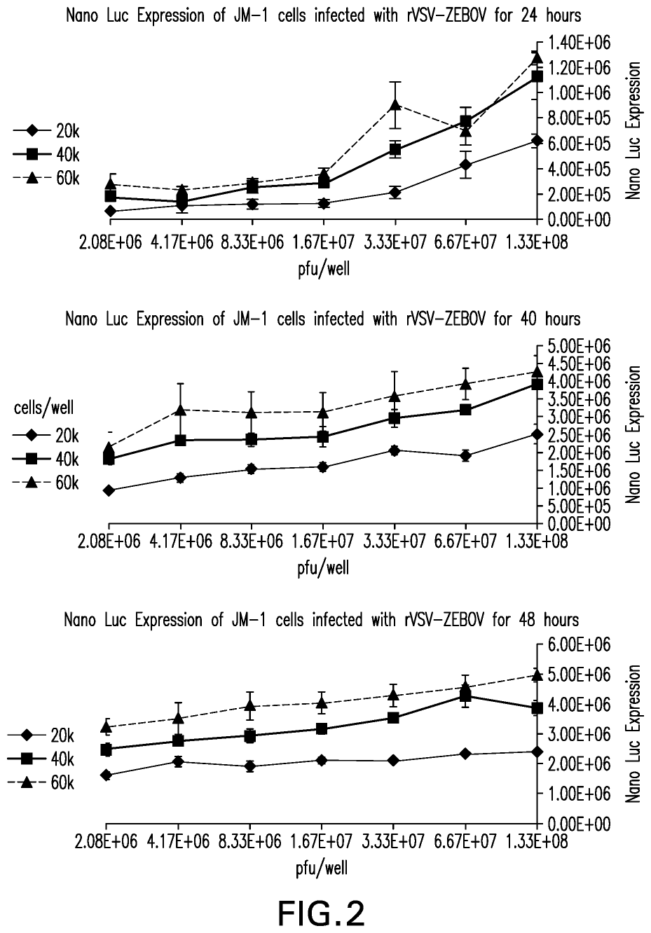

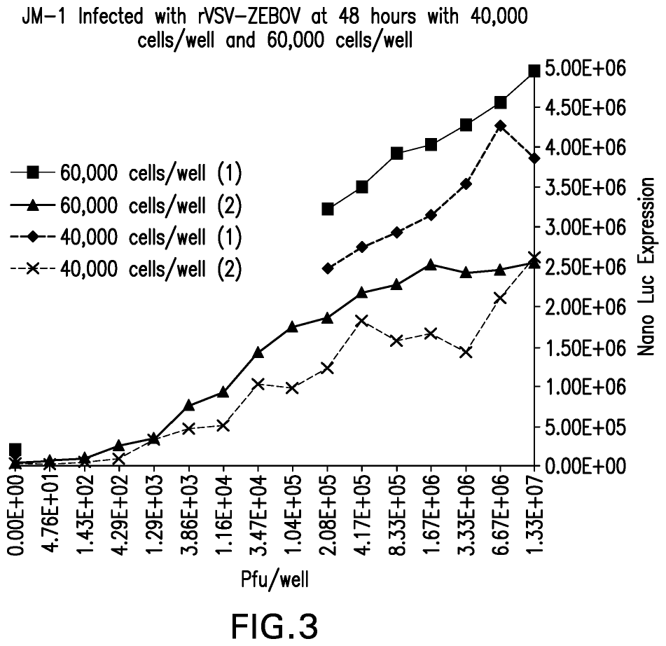

A number of experiments have been performed with rVSV-ΔG-ZEBOV-GP and the VeroE6/CMV-NanoLuc® cell lines. The first study was a titration experiment in which increasing amounts of rVSV-ΔG-ZEBOV-GP virus in a culture with VeroE6/CMV-NanoLuc® cells resulted in a concomitant increase in the measured NanoLuc® enzyme luminescent signal (FIG. 1). Following this observation, an optimization study was performed to determine the target infection time and density of cells/well with rVSV-ΔG-ZEBOV-GP and VeroE6/CMV-NanoLuc®. Evaluation of NanoLuc® enzyme expression at 24, 40, and 48 hours post- infection with rVSV-ΔG-ZEBOV-GP using VeroE6/CMV-NanoLuc® cells at a density of 20,000, 40,000, and 60,000 cells/well was performed. It was determined that an infection time of 48 hours with a cell density of 60,000 cells/well had the best linearity (FIG. 2). Because an infection time of 48 hours with a density of 40,000 cells/well also showed linearity, a titration experiment to 0 PFU/mL with rVSV-ΔG-ZEBOV-GP was performed and it was confirmed that the best linearity, signal-to-noise ratio, and dynamic range was observed with an infection time of 48 hours with a cell density of 60,000 cells/well (FIG. 3).

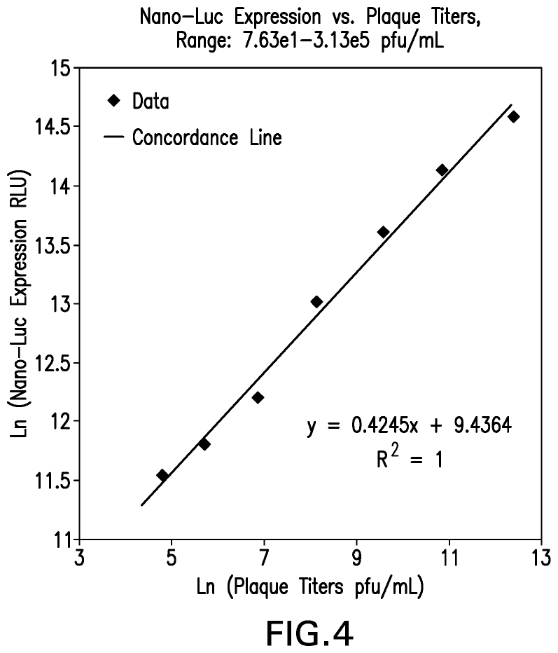

The next study focused on determining if the VeroE6/CMV-NanoLuc® cell line with rVSV-ΔG-ZEBOV-GP correlated to the plaque potency assay, which is the established assay for measuring viral infectivity/potency. From a reference standard set, (a reference standard, range 7.63E1 to 3.13E5 PFU/mL was generated and tested in both the VeroE6/CMV-NanoLuc® cell-based reporter assay and plaque potency assay in parallel) samples were diluted and evaluated in both the Vero E6/CMV-NanoLuc® and plaque potency assays. The results of this experiment are depicted in FIG. 4. The natural logarithm was obtained for each data set and plotted. It was determined that there was a correlation between the cell-based and plaque assays (FIG. 4). Attributes of the cell-based reporter assay and plaque potency assay are summarized in Table 1; which demonstrates the advantages of the cell-based reporter assay.

VeroE6/CMV-NanoLuc ® cell-based and plaque potency assays

| | VeroE6/CMV-NanoLuc | |
|---|---|---|
| | “Cell-Based Reporter | |
| Reagent/Item | Assay” | Plaque Potency Assay |
| Tissue Culture Dish | 96-well plates, n = 3 | 288 individual culture |
| | | dishes |
| Detection Reagents, | Furimazine, <2 mL | Coomassie blue, mL |
| volume | | quantities |
| Measurement | Automated/Luminometer | Manual counting with a |
| | | light box |
| Total assay time and | 1 week | Multiple weeks |
| data reporting | | |

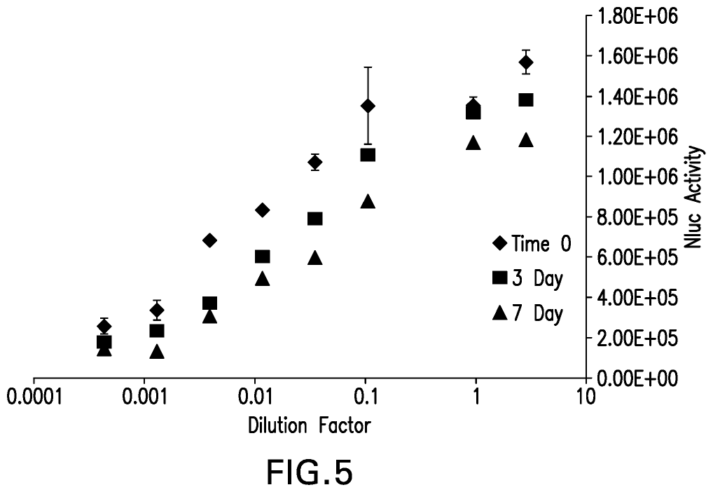

To determine if the Vero E6/CMV-NanoLuc® cell line was stability-indicating rVSV-ΔG-ZEBOV-GP samples incubated at 25° C. at 0, 3, and 7 days were evaluated. Each sample was diluted and used to infect Vero E6/CMV-NanoLuc® cells. It was determined that the Vero E6/CMV-NanoLuc® cell line was stability-indicating due to the observation that at Time 0, NanoLuc® enzyme in the media was at the highest levels as a result of the virus being intact. At 3 days, less NanoLuc® enzyme was observed in the media, and at 7 days, the lowest amount of NanoLuc® enzyme in the media was observed. The lowering of the NanoLuc® signal indicates that the virus appears to be degrading over time (FIG. 5). Using the current plaque assay, a similar degradation profile was also observed over the course of 7 days with incubation at 25° C. These results also demonstrated that the same rank-order was obtained for the Time 0, 3, and 7 day samples in both the cell-based reporter assay and plaque potency assay.

One commercially available kit to evaluate cell viability is the CellTiterGlo® Assay. Because lytic viruses result in cell rupture, or death, the viability of uninfected cells can be measured with this assay, and therefore the potency of the virus can be evaluated. Following infection (with rVSV-ΔG-ZEBOV-GP, for example), the remaining living cells are lysed with the CellTiterGlo® reagents (lysis buffer, luciferase, and luciferin substrate) wherein adenosine triphosphate (ATP) is released from the cell. The ATP from the cell is used with the luciferase substrate in a reaction with the luciferase enzyme to release light.

For the NanoLuc® assay, Vero E6/CMV-NanoLuc® cells were infected with the same titer in each well of a 96-well plate in duplicate plates with rVSV-ΔG-ZEBOV-GP. For the CellTiterGlo® assay, Vero cells were infected using the same procedure. Media was removed from the Vero E6/CMV-NanoLuc® culture and cell lysate generated from the Vero culture was mixed with either the furimazine substrate or luciferase reagents, respectively, and measured on a luminometer. Graphical representation of the data for each set of duplicate plates were averaged.

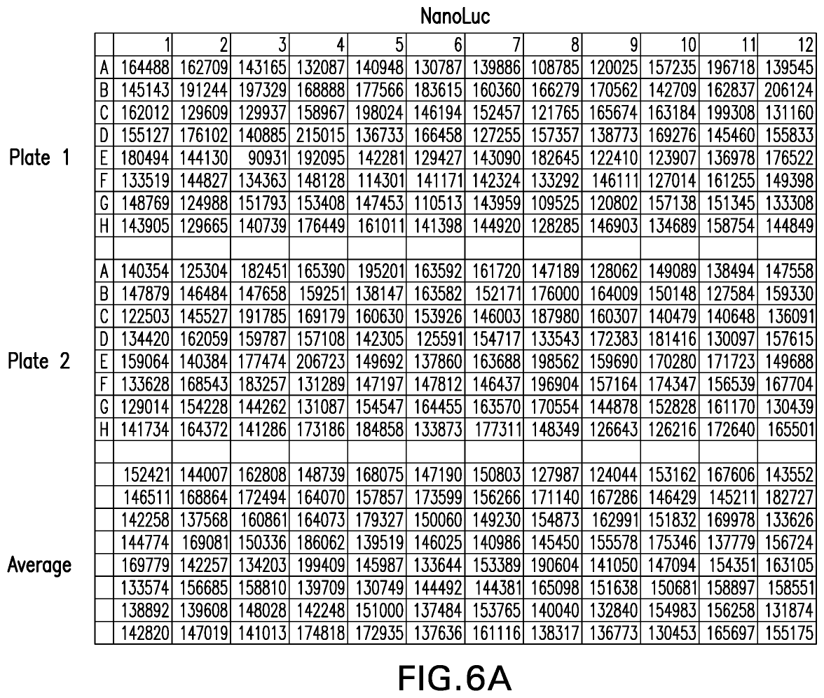

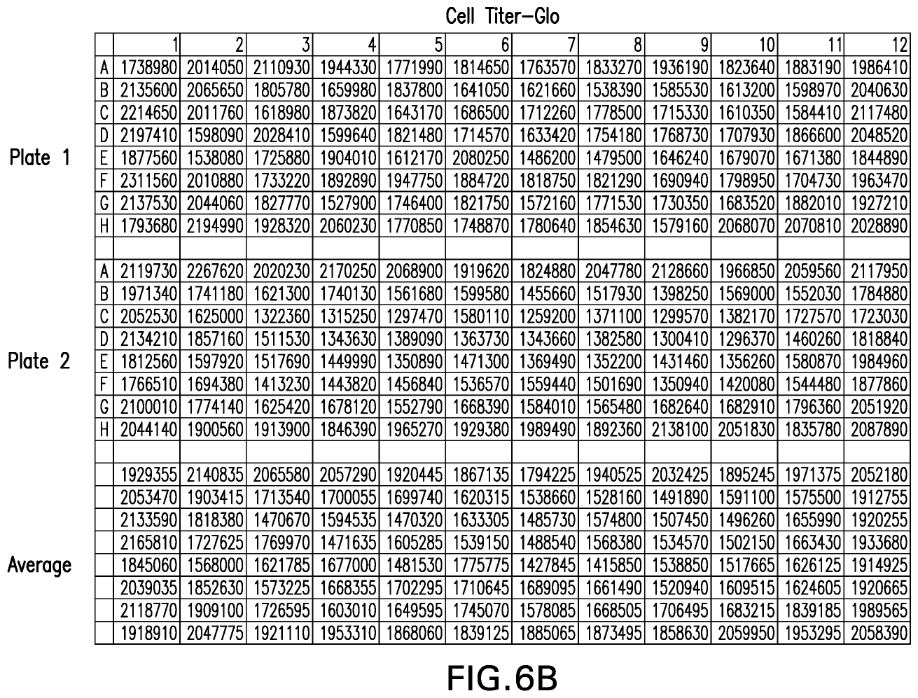

The NanoLuc® assay was determined to be random and had no edge bias whereas the CellTiterGlo® assay showed bias and had edge effects. (Dark shading indicates the highest readings and lighter shading indicates the lowest readings. For the NanoLuc® assay plates, the dark and light shades are distributed randomly suggesting no bias in the plate. For the CellTiterGlo® plates, the dark, or high values, are shown on the edges of the plate and the light, or lowest values, are in the center of the plate. Dark and light values are not random (light in the center, dark on the edge), which indicates bias in the plate; FIG. 6).

Importantly, the cell-based reporter assay of the instant invention (the NanoLuc® assay) may be used in a 96-well or 386-well format. The advantage of this format is that the total assay time is substantially lower than the conventional plaque assay (1 week vs. multiple weeks). The use of the 96-well format or the 386-well format also allows for integration with a high throughput approach.

### Example

The cell-based reporter assay of the present invention includes the ability to measure the viral potency of the rVSV-ΔG-ZEBOV-GP virus. The reporter gene is comprised of the NanoLuc® gene and is linked to a cytomegalovirus (CMV) promoter. The CMV promoter is constitutively on, resulting in generation of the NanoLuc® gene product, and ultimately, accumulation of the NanoLuc® enzyme in the cell.

Following infection of the VeroE6/CMV-NanoLuc® cell by rVSV-ΔG-ZEBOV-GP, the NanoLuc® enzyme is released into the media. Following release of the NanoLuc® enzyme into the media, the media is removed, combined with the enzyme's substrate, furimazine, and the signal measured on a luminometer. It is hypothesized that release of NanoLuc® into the media is due to lysis of the cell. Based on this hypothesis, this reporter cell line has the potential to be used with other live viruses.

Standard cell-based assays with reporter cell lines require that the cells be treated with a reagent, or infected with a virus. Following this, the cells are typically lysed and the reporter enzyme measured (with a luminometer, for example).

Conversely, for the VeroE6/CMV-NanoLuc®/rVSV-ΔG-ZEBOVGP assay of the invention, following infection with a virus, the reporter enzyme (^{NanoLuc}®) is measured in the media and lysis of the cell using a reagent is not required.

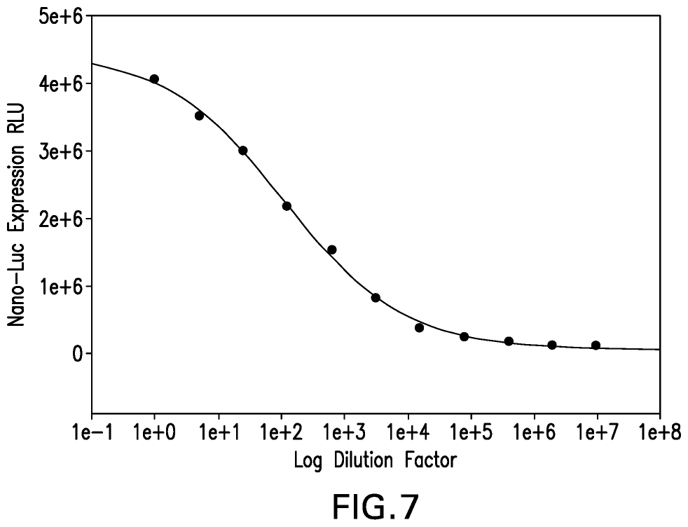

The cell-based reporter assay with VeroE6/CMV-NanoLuc® and rVSV-ΔG-ZEBOV-GP was performed in a 96-well plate. A reference standard as well as 3 samples per plate were evaluated using an 11-point curve. The 11-point curve was generated by first serially diluting the virus, then transferring the diluted virus to a 96-well plate containing Vero E6/CMV-NanoLuc® cells (FIG. 7). After 48+/-6 hours, media from the wells was removed, mixed with furimazine, and evaluated on a luminometer. The data was plotted and using a 4-parameter fit, the ECso of both the reference curve and samples were obtained. The % response for each sample was obtained by dividing the sample EC₅₀ by the reference EC₅₀ and multiplying by 100. An example of an 11-point curve that was generated with VeroE6/CMV-NanoLuc® infected with rVSV-ΔG-ZEBOV-GP is depicted in FIG. 7.

A Variance Component Analysis (VCA) was performed to determine the variability of the assay. Both a positive control sample as well as a stability sample were tested. For each plate, 3 samples, 3 positive controls, and 2 references were used. There were a total of 3 plates per run, and three runs total. Table 2. summarizes the results of the VCA.

TABLE 2
Variance components analysis, ANOVA
results generated for intra-plate, inter-plate,
run-to-run, and assay variability.
Variance Components Analysis, ANOVA

| | |
|---|---|
| Rep-to-Rep (Intra-Plate) | 22% |
| Plate-to-Plate (Inter-Plate) | 18% |
| Run-to-Run | 5% |
| Assay Variability | 29% |

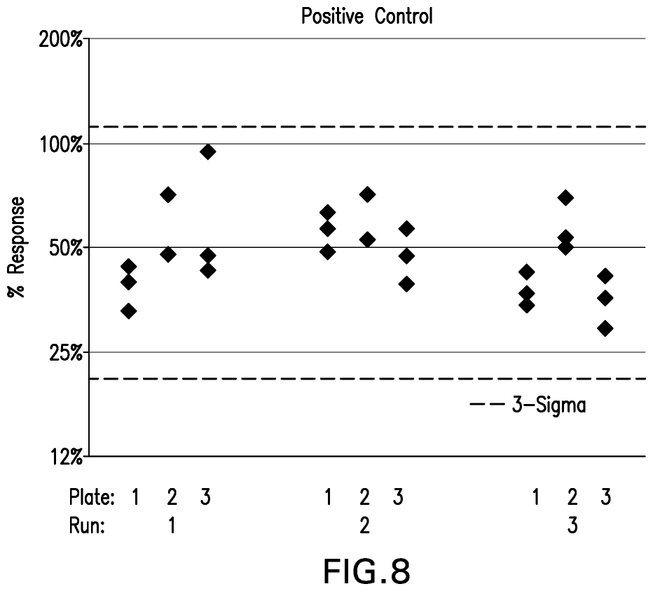

A 3 sigma analysis was also run with the positive control and it was determined that the assay range for the positive control is 21% to 111%. The range is depicted in FIG. 8.

Parallelism was also evaluated for the assay and the results are summarized in Table 3.

TABLE 3
Parallelism analyzed by obtaining the mean, standard deviation,
and coefficient variation among all 27 data sets of positive
control and all 27 data sets of sample.

| | PC Mean | SD | % CV | Sample Mean | SD | % CV |
|---|---|---|---|---|---|---|
| Parallelism | 0.84 | 0.17 | 21% | 1.05 | 0.29 | 25% |

A sample protocol for a cell-based reporter assay of the invention is described as follows:

### ABBREVIATIONS

### BSC-Bio Safety Cabinet

### DPBS-Delbecco's Phosphate Buffered Saline

EDTA- Ethylenediaminetetraacetic acid

### EMEM-Earle's Modified Eagle's Medium

### FBS-Fetal Bovine Serum

### G418-Geneticin

### PBS-Phsphate Buffered Saline

pfu-plaque forming units

Vero E6/CMV-NanoLue cells were maintained in a flask containing EMEM, 200 mM L-glutamine, 10% FBS, 1 mg/mL G418. For plating into 96-well dishes, cells were rinsed with Dulbecco's PBS without Ca²⁺ and Mg²⁺ (DPBS) and trypsinized with Trypsin-EDTA. Cells were centrifuged at 130Xg for 10 minutes and the supernatant removed. Cells were re-suspended in EMEM, 10% FBS and counted. Each well of the 96-well dish (black-walled, clear-bottom dish) was seeded with 60,000 cells and supplemented with EMEM, 10% FBS to generate a volume of 300 μl per well. The dish was incubated at 37° C., 5% CO₂.

Following incubation of the cells for 24 hours at 37° C., 5% CO₂, the cells are inspected under the optical microscope. Following inspection of cells in each well, a new serial dilution plate is created. The serial dilution plate will contain a reference standard (for example rVSV-ΔG-ZEBOV-GP), positive control, and 5 samples to be evaluated. Pipette 1674 of EMEM, 10% FBS into columns 2 through 11 . Pipette 2504 μL of EMEM and 10% FBS into column 12. Put 250 μL 3.13×10⁵ pfu/mL rVSV-ΔG-ZEBOV-GP reference in column 1. Pipette 83 μL from column 1 into column 2, 83 μL from column 2 into column 3, 83 μL from column 3 into column 4, 83 μL from column 4 into column 5, 83 μL from column 5 into column 6, 83 μL from column 6 into column 7, 83 μL from column 7 to column 8, 83 μL form column 8 to column 9, 83 μL from column 9 to column 10, 83 μL from column 10 to column 11.

Infect cell plates with the viral dilutions made in the serial dilution plate. Remove all media from the plate containing cells. Add 200 μl of culture media to each well that contains the cells. Pipette 100 μL from the column 1 serial dilution plate into the column 1 cell plate containing media. Transfer carefully to mix. Repeat for columns 2-12.

Incubate the infected cell plates in the 37° C., 5% CO₂ incubator for 48 hours.

Following the infection of cells with rVSV-ΔG-ZEBOV-GP, the media will be assayed for the presence of the NanoLuc® enzyme. Next, place the virus infected cell plates under the BSC with the light off for 15 minutes and then calculate the amount of the Nano-Glo®:DPBS Solution needed in a 1:50 ratio. Next, put white Teflon tape on the bottom of a new plate and add 1004 of Nano-Glo®:DPBS to each well with 12-channel pipette. Then, remove 1004 media from the cell plate and add to Nano-Glo®:DPBS plate, pipette up and down to mix (the plate layout will be the same as infection) and then measure on the luminometer.

### REFERENCES

Baer and Kehn-Hall, 2014. Viral Concentration Determination Through Plaque Assays: Using Traditional and Novel Overlay Systems. Journal of Visualized Experiments (93).

Cooper, P. D. 1961. The plaque assay of animal viruses. Adv. Virus Res. 8, 319-378.

Dulbecco, R. & Vogt, M. 1953. Some problems of animal virology as studied by the plaque technique. Cold Spring Harb. Symp. Quant. Biol. 18,273-279.

Hartley, J. W. & Rowe, W. P. 1963. Tissue culture cytopathic and plaque assays for mouse hepatitis viruses. 16i Proc. Soc. Exp. Biol. Med. Soc. Exp. Biol. Med. N. Y. 113, 403-406 (1963).

Li, Q, Maddox C, Rasmussen L, Hobrath J V, White L E. 2009. Assay development and high-throughput antiviral drug screening against Bluetongue virus. Antiviral Research 83, 267-73.

Masser A E, Kandasamy G, Kaimal J M, Andréasson C. 2016. Luciferase NanoLuc as a reporter for gene expression and protein levels in Saccharomyces cerevisiae. Yeast. 33(5):191-200.

Mitsuki Y Y, Yamamoto T, Mizukoshi F, Momota M, Terahara K, Yoshimura K, Harada S, Tsunetsugu-Yokota Y. 2016. A novel dual luciferase assay for the simultaneous monitoring of HIV infection and cell viability. J Virol Methods. May;231:25-33.

Niles A, Noah J, Rasmussen L and Lazar D. Determine Viral-Induced Cytopathic Effect Using a Luminescent Assay. Promega Corporation Web site.

http://www.promega.com/resources/pubhub/determine-viral-induced-cytopathic-effect-using-a-luminescent-assay/ Updated December 2013. Accessed 15 Nov. 2016.

Noah, J. W., Severson, W., Noah, D L, Rasmussen, L, White, E L, Jonsson, C B. 2007. A cell-based luminescence assay is effective for high-throughput screening of potential influenza antivirals. Antiviral Res. 73, 50-9.

What is claimed is:

1. A cell-based reporter assay for determining viral potency comprising A) transfecting cells maintained in media with a promoter-reporter construct and generating reporter enzyme within the cells; B) infecting cells with a live virus or live virus vaccine wherein reporter enzyme is released into media; and C) measuring reporter enzyme intensity in media.

2. The assay of claim 1 wherein the cells are selected from insect, animal, or human cells.

3. The assay of claim 2 wherein the cells are selected from Vero, Vero E6, MeWo, HEK293, CHO, MC3T3, DU145, H295R, HeLa, KBM-7, LNCaP, MCF-7, MDA-MB-468, PC3, SaOS-2, SH-SY5Y, T47D, THP-1, U87, NCI60, GH, PC12, BY-2, MDCK, A6, AB9, ARPE19, and MRC-5 cells and any modifications thereof.

4. The assay of claim 3 wherein the cells are Vero E6 cells.

5. The assay of claim 1 wherein the promoter is any constitutive promoter.

6. The assay of claim 5 wherein the promoter is a CMV promoter.

7. The assay of claim 1 wherein the reporter is SEQ ID 4 or SEQ ID 5.

8. The assay of claim 7 wherein the reporter is SEQ ID 4.

9. The assay of claim 1 wherein the live virus is selected from a filovirus, herpesvirus, paramyxovirus, arenavirus, adenovirus, rhabdovirus, flavivirus, and orthomyxovirus.

10. The assay of claim 1 wherein the live virus vaccine is selected from a filovirus, herpesvirus, paramyxovirus, arenavirus, adenovirus, rhabdovirus, flavivirus, and orthomyxovirus vaccine.

11. The assay of claim 9 wherein the live virus is a filovirus.

12. The assay of claim 10 wherein the live virus vaccine is a filovirus vaccine.

13. The assay of claim 1 wherein the live virus is a rVSV-ΔG virus.

14. The assay of claim 1 wherein the live virus vaccine is a rVSV-ΔG virus vaccine.

15. The assay of claim 1 wherein the live virus is rVSV-ΔG-ZEBOV-GP virus.

16. The assay of claim 1 wherein the live virus vaccine is the rVSV-ΔG-ZEBOV-GP virus vaccine.

17. The assay of claim 1 wherein the media is in liquid or gel form.

18. The assay of claim 1 wherein the media is in liquid form.

19. The assay of claim 1 wherein the assay is performed in a 96-well plate or a 384-well plate format.

20. A cell-based reporter assay for determining viral potency comprising A) transfecting Vero E6 cells maintained in media with a promoter-reporter construct wherein the promoter is CMV and the reporter is NanoLuc® enzyme and generating the NanoLuc® enzyme within the Vero E6 cells; B) infecting the Vero E6 cells with a live virus which has a rVSV-ΔG backbone; C) incubating the cells for 1-3 days at approximately 37° C. and 5% CO₂ wherein the NanoLuc® enzyme is released into the media; D) removing media; E) mixing media with substrate, and F) measuring emitted light.

21. A cell-based reporter assay for determining viral potency comprising A) transfecting Vero E6 cells maintained in media with a promoter-reporter construct wherein the promoter is CMV and the reporter is NanoLuc® enzyme and generating the NanoLuc® enzyme within the Vero E6 cells; B) infecting the Vero E6 cells with a live virus vaccine which comprises rVSV-ΔG; C) incubating the cells for 1-3 days at approximately 37° C. and 5% CO₂ wherein the NanoLuc® enzyme is released into the media; D) removing media; E) mixing media with substrate, and F) measuring emitted light.


SEQUENCE LISTING (5 sequences)

SEQ ID NO: 1 - DNA, 1378 bp, Artificial Sequence
  NanoLuc Reporter Gene
aagcttattgattattgactagttattaatagtaatcaattacggggtcattagttcatacccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataggggcaatccggtactgttggtaaagccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattg

SEQ ID NO: 2 - DNA, 578 bp, Artificial Sequence
  CMV immediate/early enhancer/promoter
attattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 3 - DNA, 516 bp, Artificial Sequence
  NanoLuc Enzyme (DNA)
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 4 - protein, 171 aa, Artificial Sequence
  NanoLuc Enzyme (Protein)
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 5 - protein, 550 aa, Artificial Sequence
  Luc2 Enzyme (Protein)
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV